(19) United States
(12) Patent Application Publication
(10) Pub. No.: US 2021/0280278 A1
(43) Pub. Date: Sep. 9, 2021

(54) System and Method for Automated Document Adjudication Communicating Clinical Trial Activities With Preparation For eFiling

(71) Applicant: Lidia Fenech, East Garafraxa (CA)

(72) Inventor: Lidia Fenech, East Garafraxa (CA)

(73) Assignee: LIDIA FENECH

(21) Appl. No.: 16/813,296

(22) Filed: Mar. 9, 2020

(51) Int. Cl. G16H 10/20; G16H 15/00; G06Q 10/10; G06F 16/23; G06F 16/93; G06F 21/62; G06K 9/00

(52) U.S. Cl. CPC G16H 10/20; G16H 15/00; G06Q 10/10; G06K 2209/504; G06F 16/93; G06F 21/6209; G06K 9/00442; G06F 16/2379

(57) ABSTRACT

The system and method to process to identify, assess, adjudicate clinical trial documents, and a proprietary communication process with users and senders each transaction of activity up to the point of eFiling for correct documents in real time including escalation pathways as needed. The system and methodology generates a tracking document for each transaction within the system.

A secure server is used to send and receive all study documents within the system using transmission capabilities. Each document, new or updated, is tracked upon receipt using a proprietary methodology, each error identified using the proprietary adjudication is tracked and prepared for communication to the user/sender in real time. The correction attempts is predetermined within the system, and escalation pathways are implemented at specified time points. All transactions/activities are tracked and prepared for eFiling within a closed cloud based system in real-time.

### TECHNICAL FIELD

The present invention is the system and method to identify, assess, adjudicate each document and communicate via template communications. Specifically, the invention relates to the area of automated identification of documents, automated real-time assessment and review of documents, real-time adjudication with real-time communication to user and sender, and uniform regulatory compliant preparation for eFiling.

### BACKGROUND INFO

Current electronic systems employ a portal system. Users require login details. This proves problematic as there are multitudes of login/password details required. Users typically have as few as 2 system logins and depending on the work and vendors used, can be upwards of 5-8 systems for login. The ability to remember each login poses a challenge. If login/password details are written down, or typed into a document, system hacks are possible, and compromises the validity of the system and the data contained.

Users must send documents to portals to upload and download documents. These portals are used to exchange, upload and download documents. The ability to maintain current required documents requires users to continuously update and reload new documents. This can be problematic if the portals are not updated regularly and in real-time. Using outdated or incorrect documents leads to delays and frustration for the user and sender.

Users apply commercially available tools to “compare” documents. The ability to compare documents will provide some limited insights. The ability to identify errors is not done. The “Compare” function allows for differences to be identified between documents. The differences identified are only different. Users must review the identified differences, and confirm if errors exist relative to the original documents. Communication must be made to staff to update with the issues listed.

Users must open systems or portals for notifications. Notifications are sent however, ongoing follow up is required for users to intervene with no set intervals identified. Notifications require user intervention which can be inconsistent, or delayed or not done at all until significant delays are noticed.

Users send emails. Users will generate email correspondence to communicate with staff. The user and sender must keep on top of all email correspondence to ensure follow up is done. If no follow up is done, it is usually left until an issue is identified as a delay.

Users can use message boards for questions, issues, communication. Message board used for issues, and/or general communications can be problematic. Staff may not enter into portals/message boards consistently to know key details. Message boards may not provide insights specific to one staff/or site but more generalized information, and staff may not pay attention to the information shared.

Dashboards, email notifications, spreadsheets and checklists are used extensively however still require user and staff intervention to complete activities and tasks.

### SUMMARY OF INVENTION

One embodiment is the system and method to process the identification of, assessment of and adjudication of documents, and a template system for communicating with users and senders up to the point of eFiling for correct documents, all in real-time. The system and methodology that tracks each transaction and prepares for compliant eFiling in real-time.

### BRIEF DESCRIPTION OF DRAWINGS

FIG. 1 is an overview of the system, technology that can be used that will enable the methodology of the system to provide the embodied functions.

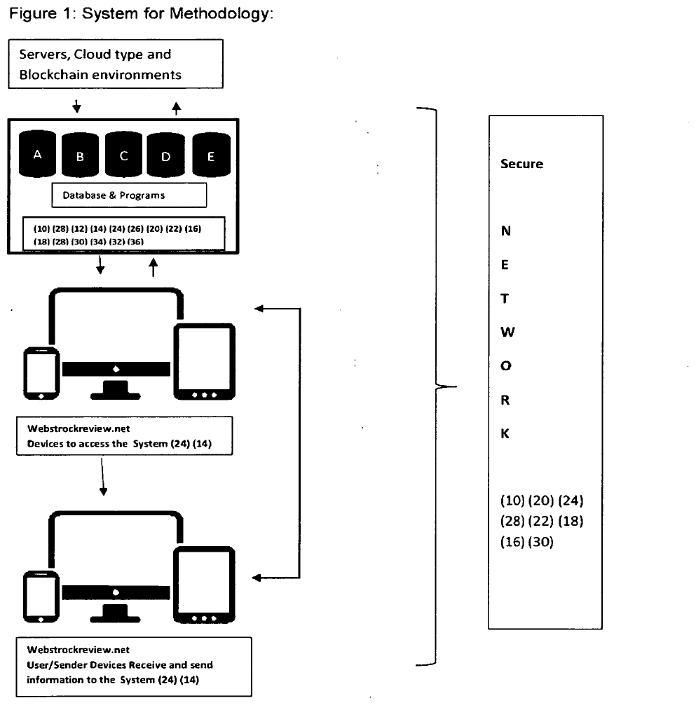

FIG. 2 is a process flow diagram of the functionality of the system and methodology within the system used to enable the identification, assessment adjudication and preparation for communication, tracking and filing of each activity and transaction, in accordance with the embodiments, all in real-time.

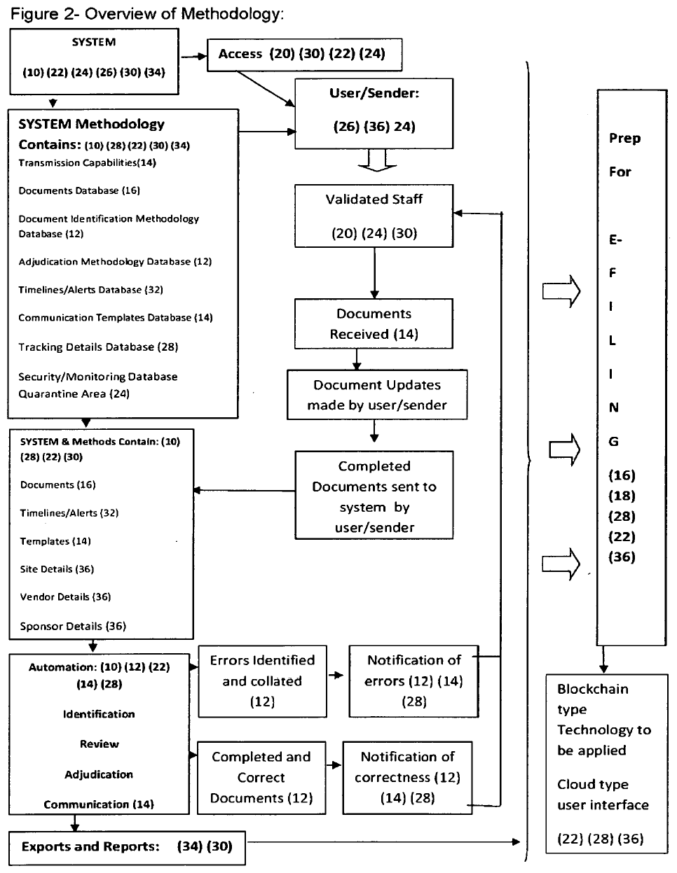

This figure also illustrates the various databases within the present invention for the collection and storage of information, and building information to enable ongoing evaluations, identification of documents in accordance with the embodiments all in real-time.

This figure illustrates the collation the documents in preparation to send to the User/Sender in accordance with the embodiments.

This figure illustrates the preparation for eFiling after activities are conducted and completed.

### DESCRIPTION OF EMBODIMENTS

One embodiment is the system and method using and building multiple databases that contain documents for use, adjudication methodology, templates for communication, tracking methodology for documents for each activity/transaction with communication capabilities built-in for real-time processing. Within the system, the documents are available for use by any qualified user with permissions.

One embodiment is the methodology applied using robotic processing type automation to complete each transaction listed in real-time.

One embodiment is the methodology to apply cloud-type infrastructure if needed for filing and storage of information as collected within the system.

One embodiment is the ability to use a computer processor, databases, cloud, mobile phone/device-type technology, or tablet-type technology to access the system, provided the qualified user has permissions granted.

One embodiment is the methodology applied which may use blockchain-type technology for security and filing purposes.

One embodiment is the ability to view all activities/transactions for a particular sponsor/study/site or any combination within the system based on being a qualified user with permissions, with the ability to export/download into a report using Microsoft-type programs as/if needed.

One embodiment is the identification of documents upon receipt in the system from a User/Sender based on a pre-determined methodology in real-time.

One embodiment is the assessment of each document within the system using proprietary algorithm and robotic processing-type automation in real-time.

One embodiment is the adjudication of each document and each field within the document based on a proprietary algorithm and robotic processing-type automation in real-time.

One embodiment is the collation of errors, with tracking of each error by form/document and field within the document and preparing for communication using robotic processing-type automation within the system in real-time.

One embodiment is the preparation of a proprietary template communication document using robotic processing-type automation, sending same communication and tracking of the communication, with preparation for eFiling of the communication in real-time.

One embodiment is the collation of correct documents with tracking of the correct specific document, preparing for communication within the system in real-time.

One embodiment is the preparation of a proprietary template communication document confirming correctness, sending same communication and tracking of the communication, with preparation for eFiling of the communication in real-time.

One embodiment is the ability to collate each activity of sending, receiving, identification of, assessment of, adjudication of each field within the document/document, each error and/or each correct field within the document/document, in a tracking system using robotic processing-type automation, with a minimum of date, time, document, user name, sender name, sponsor/study/site, field of error, document of correctness, however additional tracking may be collected as needed. All these activities/transactions are conducted within the system, where the specific databases are continually populated with newly tagged information as it is collected.

One embodiment is the collation of documents prepared for and eFiled in a pre-determined manner, using robotic processing-type automation in real-time.

One embodiment is the preparation for document migration/extraction if needed from the central main repository/database.

One embodiment is document management within the system, which is segregated by sponsor/study/site/and by document type, using robotic processing-type automation, in real-time.

One embodiment is the ability to export the tracking document into a report in real time, which may be in a Microsoft-type document and may be a pre-determined type of report with pre-determined fields based on the export type. Each transaction of exporting of a document will be tracked using robotic processing type automation and also available for export, minimal data will be collected in the export document and tracked, but not limited to, date, time, sponsor/study/site, user name, sender name.

One embodiment is the ability to export any document sent or received in real-time for review, extraction, or other such activities using robotic processing-type automation. Each transaction of export will be tracked using robotic processing type automation and also available for export, minimal data will be collected in the export document and tracking, but not limited to, date, time, document type, sponsor/study/site, user name, sender name.

One embodiment is the ability for the system and methodology to follow up based on pre-determined timelines for continuous real-time review to specified users with notifications sent, tracked and filed.

One embodiment is the ability to continuously identify, assess, adjudicate with communication capabilities 24 hours per day/7 days per week, in real-time, within the system. Follow up as pre-determined using proprietary templates, and employing a pre-determined and defined escalation pathway will be done as needed. Databases within the system will collate continuously newly updated user/sender/sponsor and site information for continued real-time processing.

One embodiment is the ability to escalate to users/sponsors if pre-determined and defined delays are triggered based on pre-defined and determined timelines.

One embodiment is the ability to use cloud-type technology to house the databases, eFiled documents and tracking documents.

One embodiment is the ability to use a secure network to enable the system and methodology to function.

### DETAILED DESCRIPTION OF INVENTION

In the following description, for explanation purposes, specific details are set forth in order to provide a thorough understanding of embodiments of the invention. However, it will be apparent that various embodiments may be practiced without these specific details. The figures and descriptions are not intended to be restrictive.

The system depicted in some of the figures may be distributed in one or more components of the system and distributed across one or more networks in a cloud type computing system, and/or blockchain type environment.

The System (10). The present invention replaces a manual process of review of study specific documents received from staff senders to an automated real-time identification of documents, automated real time review and automated real time adjudication within the present invention done 24 hours per day 7 days per week. The present invention through automation will identify, review, adjudicate in real-time and collate information to communicate document correctness or identify errors. Each error identified will be communicated and returned to the sender for correction. Once corrected, the sender returns to the corrected document within the present invention. They are assessed and once deemed correct, documents are prepared for eFiling and correct documents are communicated to staff senders. The completed correct documents will be available for review and accessible in a storage technology platform embodiment.

Platform(10). The present invention will be deployed over secure internet/network, and available for internal intranet use. The present invention will be visible by only authorized users from various platforms ie. Desktop, laptop, tablet or mobile phone with internet capabilities. The present invention can be deployed as a stand-alone proprietary system if such configuration is requested.

Applicability (26). The present invention is predominantly expected to be implemented and used in clinical research industry and it may have alternative future uses in the finance, government, academic and other healthcare industries, institutions or settings.

Access (20). The present invention allows for sponsor and study specific access based on an authorized verified email and IP address. The present invention provides secure firewalled segregated sponsor-specific study-specific silos of documents and data. The present invention receives documents from a staff sender for review and adjudication. The present invention identifies the document sent. There is a real-time review of the document within the present invention identifying correctness or errors. The present invention will collate the information and generate a detailed proprietary template communication outlining each correction required sending directly to the staff sender. The staff corrects the document returning it within the present invention. The present invention assesses and adjudicates the document identifying correctness and error. If correct, staff senders receive proprietary template communication confirmation of correctness and preparation for eFiling. If additional errors are identified, additional detailed proprietary template communication is sent for correction. This loop continues for each required document until correct. There are limitations on the number of loops until user input and intervention is requested. The present invention will be segregated by sponsor and by study to minimize crossover of sensitive data.

Compliance (22). The present invention follows 21 CFR Part 11 compliance and Sarbanes-Oxley requirements for any financial activities. The present invention embodiments may add additional compliance as required for specified or specialized requirements as needed.

Security Features (24). The present invention will allow access for confirmed and authorized user/sender email address and IP address locations. Any non-identified staff will not be permitted to send information into the present invention. Any unknown sender will be sent notification of denied access. Verification of new staff can be done on an ongoing basis and at any time. The present invention will contain a quarantine area to house email and documents received from non-authorized user until authorized. Thus limiting the need to send duplicate documents. Blockchain type technology may be applied for added security features.

Adjudication Module (12). This module conducts the automated real time proprietary identification, assessment, review, collation of correctness or errors. Each required document will be adjudicated based on pre-determined criteria from the proprietary robotic processing automation methodology. Errors and correctness are confirmed. Each document and its issues will be collated and prepared for proprietary template communication to staff. Upon return of corrected documents, the proprietary adjudication module will review for correctness and errors. There will be loops until correct. There will be a limit to the number of loops before user intervention is required. All loops and actions within the present invention will be real-time tracked and documented for eFiling.

Communication Module (14). The present invention will communicate with staff senders directly within the present invention in real time using proprietary template communications.

Acknowledgements of listed received documents, lists of errors by document, list of correct documents, follow up communication will be sent at pre-determined timelines, frequency of follow up and number of follow ups will be pre-determined, notification of completeness will be provided once documents are deemed correct. The timepoint of user intervention will be pre-determined at various stages. Each proprietary communication will be collated, tracked and prepared for eFiling. A proprietary template repository will be available for standardized and customizable communications.

Audit Trail Module (28). In the present invention, each interaction from the user and/or staff sender, receipt and disposition of documents, each communication will be date/time-stamped, and prepared for eFiling. Each interaction will be documented in the audit log as a single entry. Each activity will be available for Inspection purposes.

In the present invention, updates specific to the documents will be possible on an ongoing real-time basis. The updates based on sponsor or regulatory required changes will be logged by date and time-stamped in addition to a log of who made the changes and the authorization. The go forward timepoint for the new updated documents will be tracked and documented in real-time within the system.

Document Management Module (16). Each required document will be populated in a repository within the system. The specific pre-determined criteria for completion of each document will be centralized within this module. The module will be sponsor, study, site and document specific as applicable and fully customizable. Ongoing updates will be conducted as needed with date and timestamps and user stamp for audit trail purposes.

eFiling Preparation Module (18). The present invention through automation will prepare all required documents sent and completed, all communications, each template used and retrieved for eFiling. If the staff requires eTMF filing, this can be provided as a supplement to the eFiling preparation. A separate eTMF module can be made available. The document management module within the present invention will follow industry and regulatory accepted standard nomenclature for eFiling and eTMF preparation. This module can be done using services such as Amazon Web Services

(AWS) Microsoft Azure or the like. The cloud type infrastructure is owned and organized by said service.

As an example the services may be provided under a private cloud type model in which cloud type infrastructure system is solely operated for a single organization. The organization may also opt to have an enterprise cloud type system made available to collate information for internal purposes.

Management module (30). In the present invention, the management module will ensure secure limited access, input for authorized users and staff, specific to the project and sponsor. Enhancements are enabled within the management module. The ability to customize, integrate and enhance the present invention is possible in future iterations for sponsor, regulating bodies and/or study specifics needs. All updates or enhancements will be date/timestamped and audit log applied for implementation.

Blockchain type technology and methodology may be applied to ensure security of the data and access to the data. The blockchain type technology will be organized and owned by the service provided as determined at the time.

Reporting Module (34). In the present invention, proprietary standardized reports to communicate expected key required information to understand progress, issues, timing for next steps and to assist in prompt decision-making. A proprietary reporting communication alerting system will be made available within the present invention. The reporting module can have customized reports developed specific to sponsors, sites, documents and other criteria as necessary. Communication of the reports will be made available to key stakeholders as pre-specified. All reports will be prepared for eFiling.

Alert Module (32). In the present invention, pre-determined alerts will be sent to the staff and users. Any deviations from the planned pre-determined activities, users will be notified at specific timed intervals for intervention. All alerts will be tracked and prepared for eFiling.

Additional Modules. As the present invention evolves additional modules for stand alone eTMF module, Query module, seamless present invention modules-to-sponsor integration module, site-sponsor-vendor-information storage, data storage (36), customized reporting modules will be enabled and made available and other modules will also become evident.

What is claimed is:

1. A method of adjudicating clinical trial documents comprising:
Receiving documents from users.
Identifying each document
Assessing each document individually and each field for errors or correctness
review each field on each form individually,
assess for errors on said documents,
adjudicate for errors in each document, in real-time
collate errors, or a confirmation of correctness
a proprietary template communication is generated with an error list to the user for correction, or a proprietary template communicate is generated to confirm correctness,
communication is sent to a user
preparation of reports for user use
preparation for eFiling by sponsor, study, by site, by form
each activity tracked and documented for Inspection Readiness in real-time,

2. The method of claim 1 further comprising;
where the portions of the document have been generated or provided by other users
identify each newly updated document for review
Assessing for errors on the new documents
Adjudicating for errors on the new documents, in real-time following
Adjudicating for correctness on the new document
prepare a proprietary template communication is generated with an error list to the user for correction, or a proprietary template communicate is generated to confirm correctness, in real-time
preparation for eFiling by study, by site, by form, with each activity tracked and documented for Inspection Readiness in real-time,

3. The method of claim 1 further comprising:
Identify the newly received and updated document for review,
Assessing for errors on the newly received updated documents
Adjudicating for errors on the newly received updated documents, in real-time
Adjudicating for correctness on the newly received updated documents
a proprietary template communication is generated with an error list to the user for correction, or a proprietary template communicate is generated to confirm correctness, in real-time
transmission of communication within the system to specified users for action, in real-time
preparation for eFiling by study, by site, by form/document, with each activity tracked and documented for Inspection Readiness in real-time,

4. The method of claim 1 further comprising
the tracking of each transaction or activity of receipt of documents
The tracking of each transaction or activity of listing the errors
the tracking of each transaction or activity of confirmation of correctness
the tracking of each transaction or activity of sending communications
the tracking of each transaction or activity of receipt of new documents
the tracking of each transaction or activity of preparation for efiling
each transaction of tracking is conducted in real-time

5. The method of claim 1 further comprising
preparation for eFiling by sponsor, study, by site, by form/document,

6. The method of claim 1 further comprising
The ability to generate, export and use reports for tracked documents
The ability to generate, export and use reports for received and filed documents
The ability to generate export and track each report and prepare for filing.
